(19) United States
(12) Patent Application Publication
(10) Pub. No.: US 2015/0313541 A1
(43) Pub. Date: Nov. 5, 2015

(54) METHOD AND APPARATUS FOR DETERMINING TISSUE HYDRATION

(71) Applicant: Russell Rymut, Hartland, WI (US)

(72) Inventor: Russell Rymut, Hartland, WI (US)

(21) Appl. No.: 14/740,852

(22) Filed: Jun. 16, 2015

Related U.S. Application Data
(63) Continuation of application No. 13/308,632, filed on Dec. 1, 2011.

(51) Int. Cl. A61B 5/00; A61B 5/1455

(52) U.S. Cl. CPC A61B 5/4875; A61B 5/1455; A61B 5/7225; A61B 5/7246; A61B 5/7278; A61B 2562/0238; A61B 2576/00

(57) ABSTRACT

The present invention provides a system for the measurement of a tissue hydration value in a subject comprising a microprocessor and a sensor system having a light source and a light detector. Light is projected from the light source towards the tissue of a subject. The light projected either passes through or off of the tissues of the subject and is then received by the light detector. The light detector transmits a measurement of light intensity received by the detector to the microprocessor. The microprocessor is programmed with a tissue hydration model that utilizes the measurement of light intensity to determine the tissue hydration value for the subject.

### BACKGROUND

Homeostatis of hydration is a key element in maintaining overall health, optimizing physical performance and healthy aging. Because water forms the very basis of the human body and is the key component of all bodily fluids, loss of bodily water can have a systemic impact on the body ranging from relatively minor effects such as increased heart rate, fatigue, and cognitive impairments to severe, life-threatening effects such as cerebral edema, seizures, hypovolemic shock, and kidney failure.

Hydration is an important physiologic status indicator in several applications, roughly grouped into healthcare and physical performance. Within the healthcare arena, hydration plays a key role in geriatric care, emergency room triage, nephrology, burn therapy, chronic disease management and neonatal care. The broad area of physical performance encompasses not only athletics, but also physically demanding professions such as firefighting and military service.

Despite the widely understood importance of maintaining proper bodily hydration, there remains no generally accepted means of quantifying the hydration status of humans in the wide variety of clinical, military and athletic settings. Several technologies in the marketplace or in development attempt to provide a quantitative assessment of tissue hydration but have thus far failed to gain widespread acceptance of either health professionals or physical performance experts.

Hydration assessment techniques have thus far fallen into three general categories; 1) bioelectric impedance measurements that attempt to estimate body water based on the electrical properties of the body, 2) clinical observations such as body weight, urine output, urine color, and skin turgor that attempt to provide a qualitative assessment of an individual's hydration status, and 3) laboratory measurements such as BUN and serum creatinine that provide a quantitative, though indirect, estimate of hydration. Laboratory measurements are frequently performed to supplement and confirm clinical observations.

None of the current hydration assessment techniques, however, provide a robust and reliable estimate. Bioelectric impedance is impractical for most subjects and is subject to artifact from subject movement, water compartmentalization and other model artifacts. Clinical observations are low-cost, but cannot quantify hydration status, are subject to clinician interpretation and frequently require patient history to place the observations in the correct context. Laboratory measurements require a biologic sample, are expensive, and do not provide a rapid, continual estimate of hydration.

### SUMMARY OF THE INVENTION

In one aspect, the present invention provides a system for the measurement of a tissue hydration value in a subject. In one embodiment the system comprises a microprocessor and a sensor system having a light source and a light detector. The microprocessor is operatively connected to the light source and light detector, and controls the operation of the light source light. Light is projected from the light source towards the tissue of a subject. The light projected either passes through or is reflected by the tissue of the subject and is then received by the light detector. The light detector transmits a measurement of light intensity received by the detector to the microprocessor. The microprocessor is programmed with a tissue hydration model that utilizes the measurement of light intensity to determine the tissue hydration value for the subject. The light source can be any suitable light source such as an incandescent source or a light emitting diode. Additionally the light source may comprise multiple independent individual light sources, such as multiple led's or incandescent lights.

In one embodiment the light source comprises two separate light sources. The first light source emits a wavelength of light that is highly absorbed by water. The second light source emits a wavelength is minimally absorbed by water.

In another embodiment the invention further comprises an optical filter, or filters, which filters the light produced by the light source to a desired wavelength. In one embodiment the invention comprises at least two optical filters. The first optical filter filters the light to a wavelength of light that is highly absorbed by water, and the second filter filters the light to a wavelength that is minimally absorbed by water.

In another embodiment, the system further comprises delivery optics which direct the light produced by the light source. The delivery optics can include, but are not limited to, lens filters, lenses, or fiber optic cables.

In another embodiment, the system further comprises collection optics which direct the light produced by the light source after it has passed through, or is reflected by, the tissue of a subject and delivers it to the light detector. The collection optics can include, but are not limited to, filters, lenses or fiber optic cables.

In yet another embodiment, the system further comprises diffraction optics which diffract the light produced by the light source after it has passed through, or is reflected by, the tissue of a subject, the diffracted light being directed to the light detector. The diffraction optics can include, but are not limited to, prisms, slits, and diffraction gratings.

In another aspect, the invention provides a method of determining the tissue hydration value of a subject. The method comprises providing a light source which provides a first and second light projection towards the tissue of a subject. Each light projection has a wavelength and an intensity. A light detector is also provided which detects the intensity of the first and second light projections after the light projections have either passed through, or are reflected by, the tissue of the subject. The light detector transmits a measurement of the intensity of the light projections detected to a fluid calculation system comprising a microprocessor that is programmed with a tissue hydration model. The microprocessor utilizes the measurement of the light intensities of the first second light projections received from the light detector to determine the tissue hydration value for the subject.

In one embodiment of the method, the first and second light projections are provided at different times. In another embodiment, the first and second light projections are provided at same time, each light projection's intensity alternating at a different frequency.

In another embodiment of the invention, the light detector is positioned so that the first and second light projections transmitted by the light source are transmitted in a straight line from the light source through the tissue of the subject to the light detector. In an alternate embodiment, the first and second light projections are reflected by the tissue of the subject and are received by the light detector after reflecting off of the tissue of the subject.

Other aspects of the invention will become apparent by consideration of the detailed description and accompanying drawings.

### BRIEF DESCRIPTION OF THE DRAWINGS

FIG. 1 is graph showing the absorption of hemobglobin, water, and oxygenated hemoglobin by light of different wavelengths.

FIG. 2 is a graph showing the relation of the ratio R with the change in the percentage of body mass loss in a subject.

FIG. 3 is a block diagram outlining one embodiment of the system for the measurement of a tissue hydration value in a subject.

FIG. 4 is a diagram showing an optical path for one embodiment of a transmission sensor of the present invention.

FIG. 5 is a perspective view of one embodiment of a transmission sensor of the system of the present invention.

FIG. 6 is a diagram showing an optical path for one embodiment of a reflective sensor of the present invention.

FIG. 7 is a perspective view of one embodiment of a reflection sensor of the system of the present invention.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any nonclaimed element as essential to the practice of the invention.

### DETAILED DESCRIPTION

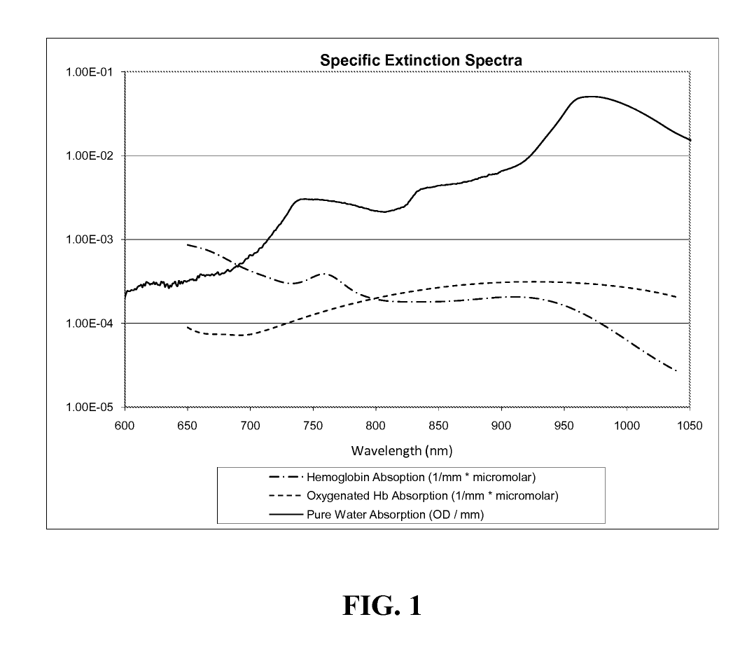

The present invention provides a method and apparatus for determining a tissue hydration value for a subject. The method and apparatus uses spectroscopic techniques to estimate a subject's hydration level. When light is transmitted through tissue, it is either absorbed or scattered in relation to the light's wavelength, the tissue's water content and the tissue's concentration of other chromophores. A specific extinction spectra is shown in FIG. 1. Both water and oxygenated hemoglobin are highly absorbing in the spectrum between 825-850 nm, but in the area around 650 nm, deoxygenated hemoglobin is the dominant absorber.

Absorption of light by a material is described by the well-known Beer-Lambert law. The law states that transmission T of light through a material is given by:

$$T = \frac{I}{{I}_{o}} = {e}^{- α l}$$

Where Iₒ is the incident light intensity, I is the intensity of light through the material, α is the material's absorption coefficient and l is the light's path length through the material.

In the presence of multiple absorbers, the total absorbance Aₜ is the sum of each absorbers independent contribution:

Aₜ=α₁l₁+α₂l₂+ . . . +αₙlₙ

Transmission of light through multiple absorbers, therefore, is given by:

$$T = \frac{I}{{I}_{o}} = {e}^{- ( {α}_{1} {l}_{1} + {α}_{2} {l}_{2} + … + {α}_{n} {l}_{n} )}$$

In the present invention, light consisting of n individual wavelengths is transmitted into the subject's tissue. The wavelengths are specifically chosen to provide at least 1 wavelength which is highly absorbed by both water and other tissue chromophores, and at least 1 wavelength which is minimally absorbed by water, but is highly absorbed by those other tissue substances.

Suitable wavelengths which are highly absorbed by water include, but are not limited to, any wavelength of the visible and near-infrared water absorption bands. Such wavelengths can be in the ranges of about 725-775 nm, about 900-1025 nm, about 1125-1225 nm, about 1350-1550 nm, or about 1850-2100 nm.

Suitable wavelengths which are minimally absorbed by water, but highly absorbed by other tissues include, but are not limited to, wavelengths in the range of about 800-825 nm, about 1025-1100 nm, about 1225-1300 nm, or about 1550-1800 nm. In one embodiment, the wavelength selected that minimally absorbs water, is selected to be relatively close to wavelength used that highly absorbs water. For instance, if a wavelength between 725-775 nm is used in one light projection, a wavelength of 800-825 nm can be used in the second light projection. Likewise the combinations of wavelengths in the range of 900-1025 nm and 1025-1100 nm, 1125-1225 nm and 1225-1300 nm, 1350-1550 nm and 1550-1800 nm, or 1550-1800 nm and 1850-2100 nm can be used.

The wavelength with high sensitivity to water is λ₁ and the wavelength with little sensitivity to water is λ₂.

Transmission of incident light of wavelength λ₂ through the tissue is given by:

$${T}_{2} = \frac{{I}_{2}}{{I}_{2 o}} = {e}^{- {α}_{c} {l}_{c}}$$

where the subscript “2” indicates that the incident light is of wavelength λ₂ and the subscript “c” indicates that the light passes through and is absorbed by the interfering chromophore.

Transmission of incident light of wavelength λ₁ through the tissue is given by:

$${T}_{1} = \frac{{I}_{1}}{{I}_{1 o}} = {e}^{- ( {α}_{c} {l}_{c} + {α}_{w} {l}_{w} )}$$

Where the subscript “w” indicates that the incident light travels through and is absorbed by water in the tissue.

The ratio:

$$R = \frac{\frac{{I}_{1}}{{I}_{1 o}}}{\frac{{I}_{2}}{{I}_{2 o}}} = \frac{{e}^{- ( {α}_{c} {l}_{c} + {α}_{w} {l}_{w} )}}{{e}^{- {α}_{c} {l}_{c}}}$$

is a value that varies greatly with respect to the path length of water encountered, but minimizes the variation with respect to the path length of the interfering chromophore. The values I₁, I₁ₒ, I₂, and I₂ₒ may be measured directly using well-known optical measurement techniques.

This raitometric technique has the advantage of minimizing the deleterious effects of both optical path length variation and variations in incident energy intensity. Non-ratiometric techniques rely on absolute measurements of optical energy, but the technique described is tolerant to variations in the optical path because these variations appear in both the numerator and denominator terms approximately equally.

The ratio R may be experimentally correlated to a clinical evaluation of tissue hydration so that ratio R can represent a tissue hydration value. Careful selection of the incident wavelengths ensures that the ratio R is approximately linear with respect to changes in hydration.

To increase accuracy and linearity and reduce the deleterious effects of other interfering chromophores, additional wavelengths may be added to the computation of R. In this case, the definition of R is extended to:

$$R = \frac{\frac{{I}_{1}}{{I}_{1 o}}}{\frac{{I}_{2}}{{I}_{2 o}} \cdot \frac{{I}_{3}}{{I}_{3 o}} … \frac{{I}_{n}}{{I}_{no}}}$$

where 2 . . . n represents the number of separate wavelengths required to provide a sufficiently accurate and linear estimate of R.

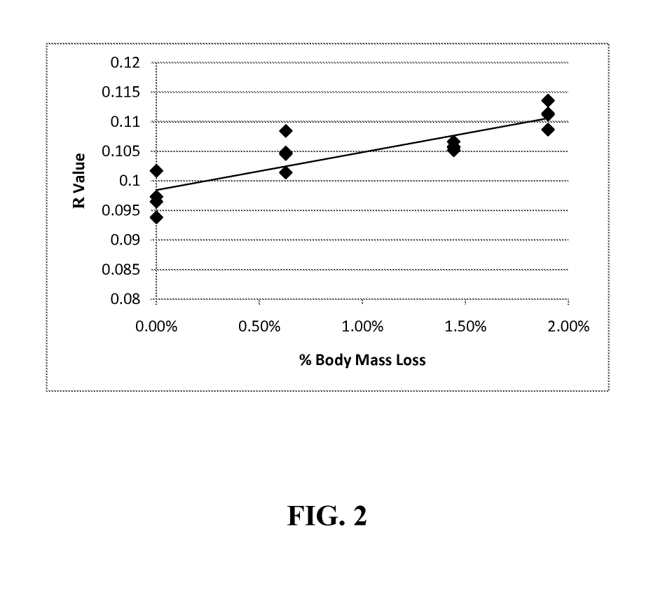

An example of the variability of R with respect to short-term weight variations (which may be used as a proxy for fluid loss) is shown in FIG. 2. In the example, λ₁≈957 nm and λ₂≈812 nm.

The ratiometric technique described is used to provide the methods and apparatus for calculating the hydration value for a subject.

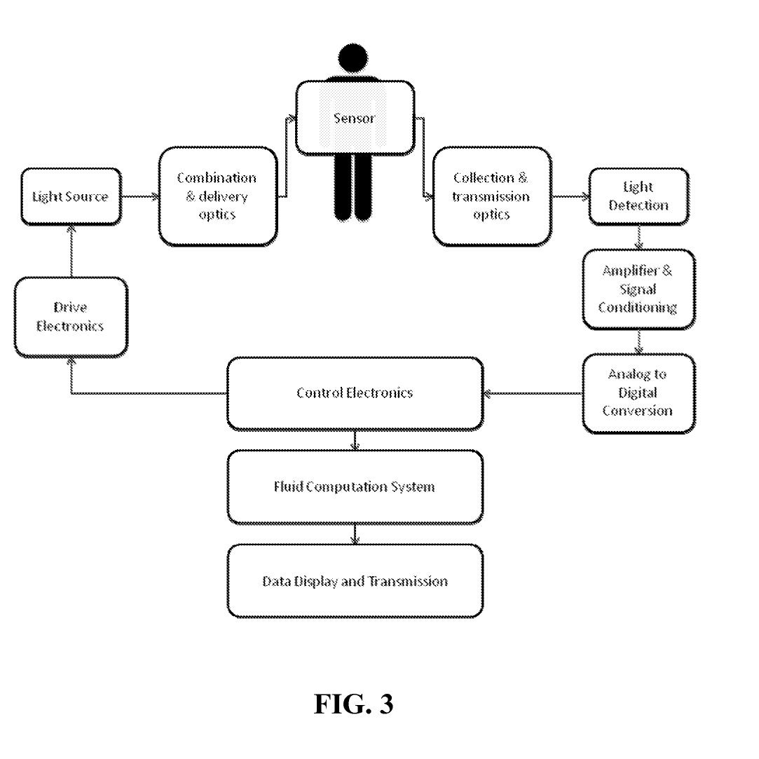

In one embodiment, the present invention provides a system for the measurement of a tissue hydration value in a subject as outlined in FIG. 3.

In one embodiment the system contains a microprocessor that runs: 1) software which controls the control electronics, which controls the various components of the system (light sources, light detectors, any delivery optics, collection optics or diffraction optics, data flow through the system and the timing associated with each act in the measurement process; 2) the fluid computation system software containing the tissue hydration model; 3) software that controls the display of values collected by the system and calculated values done by the system.

In one embodiment, the control electronics of the system consist only of the circuitry required to perform the essential measurement tasks such as the running of the light source or sources, the running of the light detectors and any necessary operation of delivery, collection or diffraction optics. Any suitable custom or commercial electronics can be used. In other embodiments, the control electronics can be integrated with, or can interface with, other external medical peripherals.

The sensor system comprises a housing which can contain the light source, the light detector, the microprocessor and the control electronics. It can also encapsulate any delivery, collection or diffraction optics, or any optional display. In alternative embodiments, any one or more of these components (light detector, microprocessor, control electronics, delivery optics, collection optics, diffraction optics, display) can be located externally from the sensor system if desired.

In one embodiment, the light source and light detector subsystem, microprocessor and control electronics are enclosed in a sensor housing. The sensor is designed to house the key optical components in such a way as to optimize light transmission from the light source through the tissue and collection of the light energy that exits the tissue to the light detector. The sensor is optimized for both body attachment and optical energy transmission.

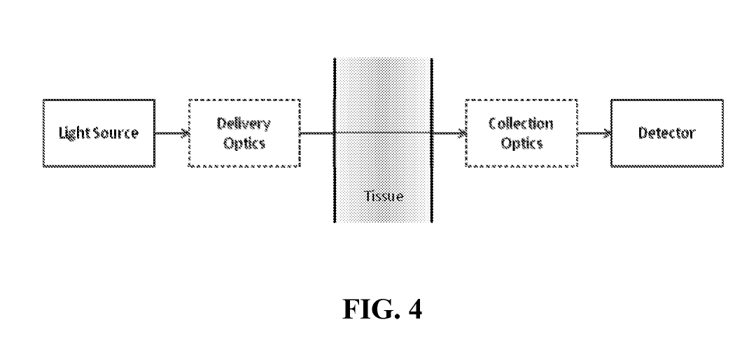

In one embodiment, the sensor attaches to the body in a location that is comfortable, accessible and provides an accurate proxy for overall fluid status. The sensor can be constructed so that, when the sensor is attached correctly, the optical energy from the light source is transmitted in a straight line from 1) the source & delivery optics, 2) through an air gap, 3) through the tissue, 4) through a second air gap, and 5) to the collection optics & detector. See FIG. 4 for a representation of one suitable optical path.

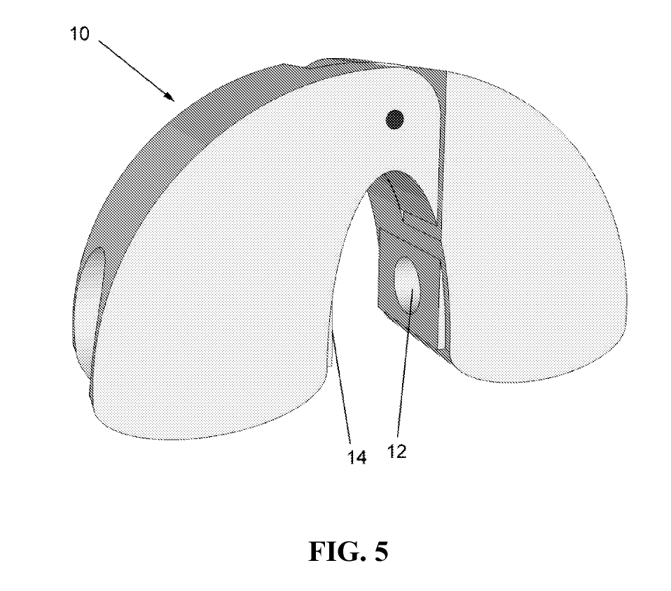

In one embodiment of the present invention, the sensor 10 (see FIG. 5) attaches to the patient's thigh and analyzes the skin, adipose and muscular tissue contained therein. The sensor is hinged to allow a fold of tissue to be contained within the sensor and correctly aligned with the light source 12 and detector 14. The sensor can be held in place by a strap that encircles both the sensor (keeping the hinge closed) and the subject's thigh (keeping the device attached to the patient).

There are numerous alternative embodiments for the sensor design:

The sensor may attach in alternate, equally suitable body locations. These locations include, but are not limited to, the lower leg, the forearm, the anterior upper arm or the posterior upper arm.

The sensor may be attached to the body via an adhesive that bonds the sensor directly to the subject's skin. The sensor may also be held in place by incorporating the sensor into a tight fitting garment that, when donned, positions the sensor correctly and keeps it in place.

Alternative sensor designs may not incorporate a hinge feature if, considering the body attachment location, the proper optical path from the light source, through the tissue and to the detector may be maintained without the feature. If required, however, the hinge may also be held closed when applied to the subject via a spring mechanism.

The optical path may include substances intended to match the index of refraction between the various optical components. Such index matching materials may be used to eliminate the deleterious effects of air gaps and other non-matched optical interfaces.

The sensor may alternately include fewer subsystems. The light source, detector and associated electronic subsystems may be located external to the sensor. In this embodiment, the sensor would include only the optical components required for optical energy delivery and collection and would be coupled to the light source and detector via fiber optic cables or other optical means. Alternatively, the sensor may contain the light source but not the light detection or contain the light detection but not the light source.

When the sensor requires an electrical interface to the system's electronics, the interface may be wired or wireless. The wireless interface would consist of commands, signals and data communicated to and from the control electronics.

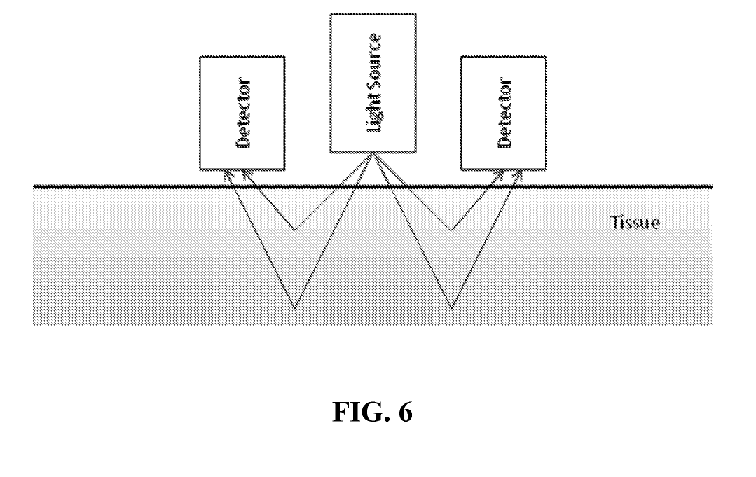

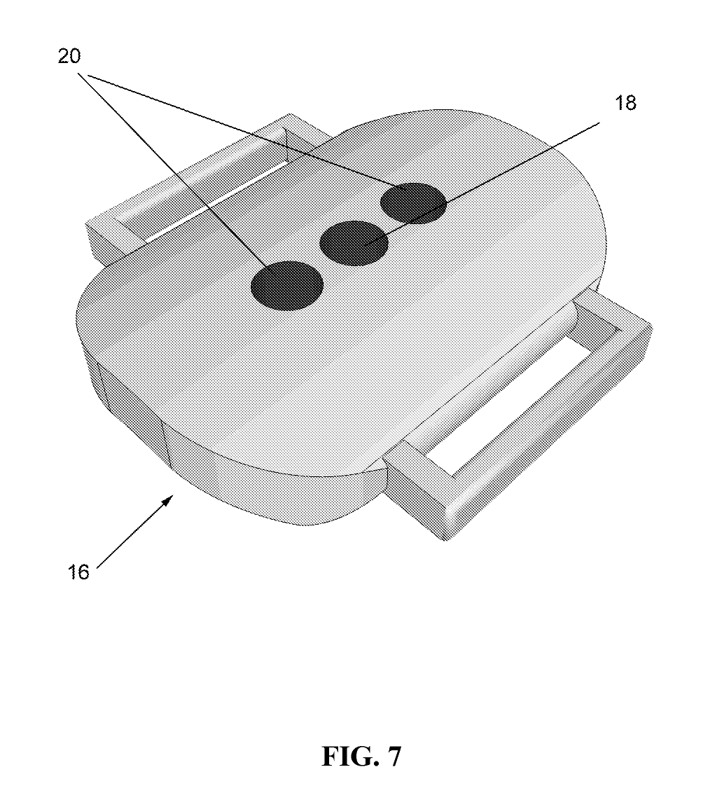

In another embodiment of the sensor, the sensor is designed to provide an optical path where the optical energy collected is energy that is reflected by the tissue instead of transmitted through the tissue. In the reflective mode, the delivery optics are on the same tissue surface as the collection optics and are positioned in such a way as to optimize the amount of optical energy collected and the path length of the energy through the tissue. The optical path of a sensor designed to operate in reflective mode is shown in FIG. 6, and an example of such a sensor is shown in FIG. 7. In the embodiment in FIG. 7, a sensor 16 houses a light source 18 and light detectors 20.

The light source is used to generate the plurality of light projections of varying wavelengths which will be absorbed by the tissue being measured. In one embodiment, the light source consists of a plurality of light emitting diodes, one for each wavelength required by the ratiometric technique. The light emitting diodes emit a spectrum of light that is sufficiently narrow to allow for the measurement technique to adequately separate the absorption of water from that of other chromophores. Alternatively, the light emitting diodes may be coupled to an optical filter in order to further narrow their optical spectrum.

In another embodiment, the light source consists of broadband light sources such as incandescent lamps. In this embodiment, the plurality of wavelengths can generated by one or more optical filters that pass the wavelengths of light required for the measurement process but reject all other wavelengths.

Regardless of type, the light source can suitably located within the sensor system. In alternative embodiments, the light source may be located remotely from the sensor and coupled to the tissue via a combination of the light source delivery optics.

In one embodiment, each specific light source is activated and powered by the drive electronics. The drive electronics consist of the circuitry required to provide sufficient and stable optical energy from the one or more light sources. The drive electronics provide the ability to turn the light sources on and off as commanded by the control electronics. The drive electronics are further capable of modulating the intensity of the light sources as required by the specific embodiments of the light sources, light detectors, and fluid computation system.

In certain embodiments, the light source can be used in combination with delivery optics. The delivery optics are intended to collect, focus and deliver the optical energy from the light source to the light detector. The delivery optics can include, but are not limited to, lenses, fiber optic cables, and mounting mechanisms that a) collect the optical energy emitted by the one or more light sources, b) combine the optical energy from the plurality of light sources into a single optical signal, and c) deliver the single optical signal to the sensor system where it is applied to the tissue being measured.

In other embodiments, much like the delivery optics, collection optics can be used to gather the optical energy that is transmitted through the tissue, focus it as required, and deliver it to the detection subsystem. The collection optics can include, but are not limited to, lenses, fiber optic cables, and mounting mechanisms that are employed to collect the exiting optical energy and deliver it to one or more light detectors.

The light detector subsystem consists of one or more light-sensitive electronic devices whose voltage or current output is proportional to the intensity of incident light. The detectors generate the estimate of the light intensity (I₁, I₂ . . . Iₙ in the calculation) to be used to calculate the index R.

In one embodiment, the light detector subsystem consists of a single silicon photodetector coupled to an amplifier and a signal conditioning subsystem. In other embodiments, different types of light detectors may be employed based on the incident wavelength of light and/or the anticipated intensity of that light. Suitable light detectors may include, but are not limited to, photomultiplier tubes, avalanche photodiodes, and InGaAs photodiodes. Other embodiments may employ a plurality of detectors, each optimized for one or more of the plurality of light sources.

In one embodiment, the light detector subsystem measures only the intensity of incident light and not its wavelength. Because of this characteristic, this embodiment relies on the plurality of light sources being time- or frequency-multiplexed.

In another embodiment, the light detector subsystem is capable of estimating the incident light's intensity at a plurality of wavelengths, allowing numerous points in the absorption spectra to be estimated simultaneously. In this embodiment, the incident light first strikes diffraction optics, such as a prism, slit or diffraction grating, that spatially separates the light according to its wavelength. The light then strikes a light detector that provides an estimate of the optical energy at a plurality of locations along the light detector's surface (a charge coupled device for example). Careful alignment of the light detector with the component providing diffraction allows estimates of the incident energy at each of a plurality of wavelengths.

In certain embodiments, the fluid detection system also comprises an amplifier and signal conditioning subsystem. The amplifier & signal conditioning subsystem consist of the electronic components required to convert, amplify, filter and otherwise condition the signals generated by the light detector subsystem. Suitable care must be taken in the design of the amplifier & signal conditioning to ensure that the required bandwidth, stability and resolution of the signal is maintained while noise and other deleterious signal components are removed.

As the signal from the light detection system may also be converted from an analog signal to a digital signal so as to allow further processing by the fluid computation system software. This is accomplished using any one of a variety of well understood techniques, many of which are available as commercially available analog to digital converter electronic components.

The calculation of R requires estimates of the optical energy incident upon the tissue, I₁ₒ, I₂ₒ . . . Iₙₒ. These estimates may be accomplished by providing an alternative optical path from light source to light detector that does not include a tissue sample. In one embodiment, the estimates of I1o, I2o . . . Ino are calculated using the subsystems as described above, but at a time during which the sensor is not attached to the subject. The control electronics subsystem manages the timing, computation and storage of the incident light estimation.

In another embodiment, the incident light energy may be directed away from the tissue under evaluation and directly to the detector subsystem using alternative embodiments of the delivery and collection optics. In this embodiment, the desired optical path (through the tissue or bypassing the tissue) is selected by the control electronics which are responsible for managing the timing, computation and storage of the incident light estimation.

In yet another embodiment, the light source directs a fixed, known proportion of the incident light energy away from the tissue under evaluation and directly to the detector subsystem at all times. In this embodiment, the detection subsystem described may be duplicated to measure the incident light estimations or a multiplexing technique may be employed to permit the same detection subsystem to measure both the incident light intensity and the transmitted light intensity.

In other embodiments, a determination of incident light is not needed, if a single light source is utilized and its overall spectrum is stable, both the ratio

$$\frac{{I}_{1 o}}{{I}_{2 o}}$$

and the ratio R remain constant regardless of the absolute intensity of the light source.

The microprocessor of the invention is programmed with a fluid computation system that can take the measurements transmitted by the light detector and translate it into an R value which is indexed against a tissue hydration model that correlates the R value to a tissue hydration value. The output of the tissue hydration model is an index that describes the fluid balance status of the subject against various R values. The index may be an estimate of an actual physical property of the subject such as total body water (TBW) or may be an estimate of hydration status on an arbitrary hydration scale.

Tissue Hydration Model for TBW

The following example will assume that the tissue hydration value is TBW, but it is understood that any suitable hydration index may be estimated using the following technique.

The fundamental component of the fluid computation system is a tissue hydration model. The model consists of a multi-dimensional space that maps a plurality of normalized input parameters to their contribution to the overall hydration status of an individual. The model is developed using a calibration data set, and is applied to a set of attributes and parameters for an individual to determine that subject's hydration index.

The calibration data set is collected on a subject population that maximizes the variability among all parameters correlated to estimation of the hydration index, such as (but not limited to) age, gender, skin color, muscle mass, fat mass, and disease state.

For each subject in the calibration population, a test protocol is executed that modulates the subject's TBW. Throughout the TBW modulation, the R value is measured simultaneously with a “gold standard” TBW measurement acquired, for example, through bioelectrical impedance analysis. See C O'Brien, A J Young, and M N Sawka, “Biolectric impedance to estimate changes in hydration status,” Int J Sports Med, vol. 23, pp. 361-366, 2002, incorporated herein in it's entirety by reference. Because some patient attributes are modulated in concert with TBW, their value is also collected simultaneously with the R values. The complete calibration data set, therefore, consists of a series of R/TBW/attribute measurement sets annotated with a variety of measurements & observations about each calibration subject.

When the calibration data set has been collected, a mathematical model linking each variable to an estimation of TBW must be determined. The model may be determined using any of a variety of well known signal processing techniques including (but not limited to) factor analysis or principal component analysis. To facilitate the model building process, non-numeric attributes of the data (such as gender) are often “coded” to allow mathematical manipulation of the attributes.

In one embodiment of the TBW model, only the value of R is required to estimate TBW to within an acceptable error limit. In this embodiment, a simple lookup table can be used to determine TBW for a specific value of R. More sophisticated mathematical models can be used to reduce the error of the TBW estimate at the cost of increased computational, calibration data, and measurement data requirements.

Estimation of an individual's fluid status, therefore, relies on the current measurement of R and a priori knowledge of the TBW model and the relevant attributes of the individual.

The measurement of R begins with estimating I₁, I₂ . . . Iₙ (the optical energy contained in the water sensitive wavelength band and other non-water sensitive wavelength bands, respectively). The method used to estimate these values varies based on the specific embodiment of the tissue hydration measurement system of the present invention, but the most fundamental implementation consideration is the method used to multiplex the plurality of source wavelengths. When multiplexed in time, the system uses the light detection subsystem to sequentially measure the optical energy at a time corresponding to the activation of the corresponding light source. These measurements are the estimates of I₁, I₂ . . . Iₙ.

When multiplexed in frequency, each light source is simultaneously modulated at a unique frequency. The light detection subsystem then measures the time-varying optical energy and I₁, I₂ . . . Iₙ are estimated utilizing one of a variety of simple frequency demodulation techniques that are well known in the industry.

When multiplexed by wavelength, the light detection subsystem must be capable of estimating the optical energy uniquely at a plurality of wavelengths. The estimates of I₁, I₂ . . . Iₙ are computed by associating the energy measurement at the wavelength that corresponds to the incident light.

When multiplexed spatially, the light detection subsystem contains a plurality of devices positioned to collect the transmitted light (I₁, I₂ . . . Iₙ) at the physical position that corresponds to the source wavelengths.

The estimates of I₁ₒ, I₂ₒ, . . . Iₙₒ are computed in a similar manner to the estimates of I₁, I₂ . . . Iₙ. In order to provide a robust estimate, however, the optical path during these measurements should optimally not include any components that would impact the relative energy measured at the plurality of source wavelengths.

Using the measurements of I₁, I₂ . . . Iₙ and I₁ₒ, I₂ₒ, . . . Iₙₒ, the hydration computation system computes the estimate of R using the simple mathematical relationship described above.

The value of R is then applied to the tissue hydration model in concert with the subject's relevant attributes. The mathematical relationship described by the tissue hydration model yields the individual's estimate of total body water (a tissue hydration value).

The algorithms encompassed in the fluid computation subsystem may be executed on the same microprocessor and electronics as the control electronics subsystem or may be partitioned to a separate microprocessor system or systems. Optimization of the microprocessor circuitry in the context of the required algorithms is a task that is well understood by those skilled in the art of microprocessor circuit design.

In one embodiment, the fluid computation system provides repeated, continuously varying estimates of R and, therefore, quasi-realtime estimates of hydration. However, alternative embodiments may provide episodic or on-demand estimates.

Once the estimate of the fluid status index is available, it may be displayed to a clinician and/or transmitted to external medical data collection systems. The display to clinicians may be via a display mounted directly to the sensor or via an external display. Communication of the fluid status index is performed using any one of a variety of well understood wired or wireless communication mechanisms, including (but not limited to) RS-232, Universal Serial Bus, Serial Peripheral Interface, Bluetooth, Zigbee or a proprietary data interface.

The display of fluid status (and the transmission of that data) may optionally include display (and transmission) of alarm status based on that data. The alarm is based on clinically significant levels of the fluid status index that indicate hyper- and/or hypo-hydration status. Alarms can provide a clinically useful indicator that the patient's fluid status is suboptimal.

Use of the Tissue Hydration Measurement System for Quantifying an Individual's Hydration Status

A variety of known technologies are currently used to estimate and quantify hydration. None of the known approaches, however, indicates to the clinician if the patient's hydration status requires intervention or not. In fact, even when a direct measurement of the individual's TBW is available, that measurement must be considered in terms of the patient's age, gender and other clinical conditions to determine if the given fluid volume is “good” or “bad”.

Clinicians observe, understand and evaluate a variety of patient parameters and observations in order to achieve a proper patient diagnosis or decide on the most effective treatment regimen. When new physiological parameters are introduced into clinical practice, a significant amount of experience must be developed before that parameter can be useful in the context of the entire patient condition. A simplified means of presenting new clinical information, therefore, is desirable.

The current invention provides a platform to generate a simplified method of assessing the set of parameters related to an individual's hydration status. A Hydration Index may be defined as a single, dimensionless parameter that is scaled such that a patient's overall hydration status can be understood from its magnitude alone. Extending the attributes included in the tissue hydration model to include additional clinical inputs, physiological parameters, and a “gold standard” clinical assessment of overall hydration (instead of Total Body Water) allows the model to provide a more complete clinical estimation of fluid status than any single fluid measurement alone.

In practice, the Hydration Index provides a simple assessment parameter that enables clinicians to understand how their diagnosis and treatment decisions may be affected by the patient's hydration status. The underlying input data (model attributes), however, are not hidden from the clinician.

Thresholds may be assigned to the Hydration Index that determine the point at which (and the level to which) a patient requires intervention for the treatment of dehydration. In addition, alternative thresholds may be assigned for other clinical uses, such as guiding medication dosing decisions.

To create the Hydration Index, the calibration data set used to generate the tissue hydration model is expanded to include additional physiological measurements and observations related to the subjective clinical assessment of hydration. Additionally, the “gold standard” hydration assessment is captured by medical and clinical experts on a quantitative, dimensionless scale allowing for its inclusion in the model. As in the tissue hydration model that estimates TBW, the calibration data set must maximize the variability among all physiological parameters and attributes included in the model.

During the calibration data collection and model building process, it is highly desirable to collect input parameters and assessments on individuals as their physiologic status changes due to disease progress, treatment progress or other dynamic conditions. It is also highly desirable to collect professional clinical opinions from a variety of sources including individuals with backgrounds in various specialties as well as a variety of individuals within each specialty.

There are many specific individual parameters related to hydration status that may be included in the Hydration Index model. The parameters include, but are not limited to: total body water, relative tissue hydration, weight, age, gender, pulse, body temperature, BUN, creatinine, blood pressure, respiration rate, urine osmolality, saliva osmolality, hematocrit, cardiac output, urine sodium, urine output.

One advantage of using the Hydration Index model of the present invention is that these parameters need not be considered singularly. The model-based Hydration Index considers a set of parameters that relate to hydration and combine them to form a clinically relevant indicator. While a minimum number of inputs are required for an accurate Hydration Index calculation, it is highly desirable that most model input parameters be optional allowing for the Hydration Index to be used in clinical environments where a only subset of model inputs are available.

Use of the Tissue Hydration Measurement System for Noninvasive Measurement of Water in Various Compartments of Tissue Through their Optical Properties

In the body, water is distributed between intracellular and extracellular compartments. Extracellular water may be further classified as intravascular water (blood plasma) and extravascular water (interstitial fluid). Water in each compartment contains a variety of solutes including sodium ions, potassium ions, glucose and many other analytes.

Physiologic dehydration can take several forms and is often classified based upon plasma tonicity. Hypertonic dehydration occurs when water is lost from the body without a corresponding loss of electrolyte. Isotonic dehydration indicates a balanced loss of water and electrolyte, and hypotonic dehydration occurs when loss of electrolyte exceeds loss of water. Each form of dehydration has differing clinical risks and treatment plans, so a clinically useful dehydration diagnosis must account for all three forms.

At homeostatis, the relative concentrations of water and analytes in each compartment are at equilibrium. However, during dynamic physiologic conditions, diffusion of water and analytes occurs across the compartment membranes as described by Fick's law. As the analyte concentration gradient across the membrane increases, water and solute diffuse across the membrane until equilibrium is established. Diffusion is the primary process by which substances contained in blood serum are distributed to the tissues that require them.

Because the membranes that define water compartments are highly permeable to water, but only moderately permeable to most solutes, even a moderate analyte concentration gradient can cause a significant amount of water to diffuse across the membrane toward the region of higher solute concentration, resulting in significant changes to cellular volume.

During dehydration the body's electrolyte status can have a dramatic effect on the relative water compartmentalization. Estimation of water compartmentalization, therefore, may be a useful indicator that aids clinicians in arriving at a proper and complete dehydration diagnosis.

Estimation of relative fluid compartmentalization can also be a key to understanding the cause and treatment of hypervolemia and hypovolemia, conditions in which the volume of fluid inside the vasculature is suboptimal. Hypervolemia and hypovolemia can occur with or without corresponding changes in TBW, so estimates of compartmentalization in conjunction with an estimate of TBW can be particularly beneficial.

Each of the various layers of tissue (stratum corneum, epidermis, dermis and hypodermis in the skin, adipose, bone and muscle in deeper tissues) have unique optical properties based on their thickness, water content, refractive index, absorption, and the size and density of scattering sites. As described above, changes in analyte concentration in response to physiologic needs causes the distribution of water throughout the various compartments to modulate. Because this redistribution can be profound, the effects on each individual tissue's optical properties can also be profound.

The tissue hydration model concept of the current invention may be extended by including a unique model that estimates the fluid contained in each clinically significant compartment. Each model may be developed as described above, but because the goal is to estimate the water contained in each compartment, both the absorption spectra of interest and the physical configuration of the optical path must be optimized for each compartment. Additionally, a “gold standard” assessment of water compartmentalization must be available within the calibration data set to accurately determine the tissue hydration models.

Because each tissue, component and analyte has, in general, different optical characteristics at a particular wavelength, additional incident light wavelengths may be employed in the calculation of R and the tissue hydration index. Each compartment may employ a unique set of wavelengths to generate I₁, I₂ . . . Iₙ, including a unique set of light sources and detectors as required.

The physical configuration of the light source and detector determines exactly which tissues are interrogated by the incident light. Careful design of the size of the incident light source, size of detector and spacing between the two determines the depth and nature of the individual compartments.

The goal of modifying the optical path to interrogate only certain tissues generally necessitates a modification of the sensor subsystem. In one embodiment, the optical path operates in the reflective mode and the sensor is designed so that light travels through the desired tissue before being reflected back to the detectors. The distance between the light source and detection subsystems can be adjusted to modify the approximate penetration depth of the incident light.

In addition to extending the tissue hydration model as described, the desire to estimate water compartmentalization provides an opportunity to include a variety of heuristic measures both within the described model and independent from it.

One set of heuristic features relates to the physiological characteristics of fluid within each particular compartment. For example, the pulsitile nature of arterial blood flow may be utilized to estimate the amount of incident light absorbed within the arterial space. Utilizing these compartment-specific values in the tissue hydration model allows the system to estimate the amount of water within the particular compartment to which the physiological feature corresponds.

An additional set of heuristic features relates to the spectral features of fluid within each particular compartment. The features of the measured absorption spectra (bandwidth of a peak, relative amplitude of a plurality of peaks, bandwidth of a trough, etc.) are determined by the various absorption, scattering and reflection properties of the tissue in each component. Measurement of these features, therefore, may be correlated to the relative optical characteristics of each compartment. Features of the first, second and higher order derivates of the spectra may be combined with the measured spectra to enhance the estimation.

Use of the Tissue Hydration Measurement System in a Body-Area Sensor Network

In many applications, it is desirable to attach a plurality of physiological sensors to an individual and allow those sensors to communicate to each other and/or a communication hub. The communication hub then transmits data from the set of sensors to a central review station. These body-area networks are particularly advantageous when utilizing a single large monitoring device is impractical, such as during firefighting. The tissue hydration monitoring system is a natural extension to body area sensor networks.

When utilized in a body-area network, the preferred embodiment of the sensor subsystem contains the light source, light detection, and all associated optical components in a single device. Additionally, the fluid computation system and control, drive, amplifier, signal conditioning and analog to digital conversion electronics are also enclosed within the sensor. Inclusion of each of these components allows the sensor subsystem to communicate the final tissue hydration estimate to the body-area network sensors and/or communication hub.

Communication of the tissue hydration index is preferably performed using a short-range wireless network. When communicating wirelessly, it is also preferred that the sensor subsystem contain a power source, enabling truly wireless operation. However, a simple wired connection may also be employed which allows distribution of power and improves communication reliability.

Generally, the sensors in a body-area network do not incorporate a display to provide the physiological measurements to the user. Rather, the data is transmitted to a central review station via the communication hub. In this manner, data from the plurality of sensors in the network may be viewed together, allowing a clinical expert to assess the subject's situation in the context of all available physiological parameters.

Various features and advantages of the invention are set forth in the following claims.

1-22. (canceled)

23. A system for the measurement of a tissue hydration value in a subject, comprising:
a microprocessor;
a sensor system comprising a light source and a light detector;
wherein the microprocessor is operatively connected to the light source and light detector; and the microprocessor controls the light source and the light detector and is programmed with a tissue hydration model that utilizes a measurement of light intensity received by the light detector from light projected from the light source after the light passes through or is reflected by a tissue of the subject to determine the tissue hydration value for the subject,
wherein the light source generates a first wavelength of light, the first wavelength of light being in one of the following ranges selected from the group consisting of 725-775 nm, 900-1025 nm, 1125-1225 nm, 1350-1550 nm, or 1850-2100 nm, and a second wavelength of light, the second wavelength of light being in one of the following ranges selected from the group consisting of 800-825 nm, 1025-1100 nm, 1225-1300 nm or 1550-1800 nm,
wherein the first wavelength of light is directed towards the tissue of the subject and, based on the measurement of light intensity from the light detector, the microprocessor determines a value for the first amount of light transmission through, or reflected off of, the tissue of the subject,
wherein the second wavelength of light is directed towards the tissue of the subject and, based on the measurement of light intensity from the light detector, the microprocessor determines a value for the second amount of light transmission through, or reflected off of, the tissue of the subject,
wherein the microprocessor uses the value for the first amount of light transmission to calculate a T₁ value, where T₁ is the value for the transmission of light of the first wavelength;
wherein the microprocessor uses the value for the second amount of light transmission to calculate a T₂ value, where T₂ is the value for the transmission of light of the second wavelength;
wherein the microprocessor determines an R value, where R=T₁/T₂
wherein the microprocessor indexes the R value against the tissue hydration model to provide a tissue hydration value, and
wherein the tissue hydration model comprises a look-up table including a calibration data set correlating a set of R values to a set of tissue hydration values.

24. The system of claim 23 wherein the sensor system further comprises delivery optics which direct the light produced by the light source, the delivery optics selected from the group consisting of lens filters, lenses, or fiber optic cables.

25. The system of claim 23 wherein the light source comprises a first and a second light emitting diode

26. The system of claim 23 wherein the light source generates the first wavelength of light by the light source comprising a light emitting diode
coupled to a first optical filter which filters the light produced by the light emitting diode to produce the first wavelength of light; and
wherein the light source generates the second wavelength of light by the light emitting diode also being coupled to a second optical filter which filters the light produced by the light emitting diode to produce the second wavelength of light.

27. The system of claim 23 wherein the light source comprises a first and a second light emitting diode;
wherein the first wavelength of light is generated by the first light emitting diode coupled to a first optical filter which filters the light produced by the first light emitting diode to produce the first wavelength of light; and
wherein the second wavelength of light is generated by the second light emitting diode coupled to a second optical filter which filters the light produced by the second light emitting diode to produce the second wavelength of light.

28. The system of claim 23 wherein the light source generates the first wavelength of light by the light source comprising an incandescent light;
the incandescent light coupled to a first optical filter which filters the light produced by the incandescent light to produce the first wavelength of light; and
wherein the light source generates the second wavelength of light by the incandescent light coupled to a second optical filter which filters the light produced by the incandescent light to produce the second wavelength of light.

29. The system of claim 23 wherein the light source comprises a first and a second incandescent light;
wherein the first wavelength of light is generated by the first incandescent light coupled to a first optical filter which filters the light produced by the first incandescent light to produce the first wavelength of light; and
wherein the second wavelength of light is generated by the second incandescent light coupled to a second optical filter which filters the light produced by the second incandescent light to produce the second wavelength of light.

30. The system of claim 23 wherein the sensor system further comprises collection optics which direct the light produced by the light source after it has passed through tissue of a subject and deliver it to the light detector, the collection optics selected from the group consisting of lenses or fiber optic cables.

31. The system of claim 23 wherein the sensor system further comprises diffraction optics which diffract the light produced by the light source after it has passed through tissue of a subject, the diffracted light being directed to the light detector, the diffraction optics selected from the group consisting of prisms, slits, and diffraction gratings.

32. A method of determining a tissue hydration value of a subject comprising:
providing a light source which provides a first light projection having a first wavelength and a first intensity, the first wavelength being in one of the following ranges selected from the group consisting of 725-775 nm, 900-1025 nm, 1125-1225 nm, 1350-1550 nm, or 1850-2100 nm; and a second light projection having a second wavelength and a second intensity, the second wavelength being in one of the following ranges selected from the group consisting of 800-825 nm, 1025-1100 nm, 1225-1300 nm or 1550-1800 nm;
directing the first light projection and the second light projection toward a tissue of the subject;
providing a light detector which detects first and second intensities of the first and second light projections, respectively, after the first and second light projections have either passed through or are reflected off of the tissue of the subject;
the light detector transmitting the first and second intensities of the first and second light projections to a fluid calculation system comprising a microprocessor that is programmed with a tissue hydration model;
wherein the tissue hydration model comprises a look-up table including a calibration data set correlating a set of R values to a set of tissue hydrations values;
the microprocessor determining a first amount of light transmission (T₁) through, or off of, the tissue of the subject based on the first light intensity from the light detector;
the microprocessor determining a second amount of light transmission (T₂) through, or off of, the tissue of the subject based on the second light intensity from the light detector;
the microprocessor determining an R value, where R=T₁/T₂; and
comparing the R value to the tissue hydration model to determine the tissue hydration value.

33. The method of claim 32 wherein the first and second light projections are directed at the tissue at different times.

34. The method of claim 32 wherein the first and second light projections are directed at the tissue at same time and wherein the first light projection has a first frequency, and the second light projection has a second frequency which is different than the first frequency.

35. The method claim 32 wherein the light source comprises a first and a second light emitting diode.

36. The method of claim 32 wherein the light source generates the first wavelength of light by the light source comprising a light emitting diode
coupled to a first optical filter which filters the first light projection produced by the light emitting diode to produce the first wavelength of light; and
wherein the light source generates the second wavelength of light by the light emitting diode also being coupled to a second optical filter which filters the second light projection produced by the light emitting diode to produce the second wavelength of light.

37. The method of claim 32 wherein the light source comprises a first and a second light emitting diode;
wherein the first wavelength of light is generated by the first light emitting diode coupled to a first optical filter which filters the first light projection produced by the first light emitting diode to produce the first wavelength of light; and
wherein the second wavelength of light is generated by the second light emitting diode coupled to a second optical filter which filters the second light projection produced by the second light emitting diode to produce the second wavelength of light.

38. The method of claim 32 wherein the light source generates the first wavelength of light by the light source comprising an incandescent light;
the incandescent light coupled to a first optical filter which filters the first light projection produced by the incandescent light to produce the first wavelength of light; and
wherein the light source generates the second wavelength of light by the incandescent light coupled to a second optical filter which filters the second light projection produced by the incandescent light to produce the second wavelength of light.

39. The method of claim 32 wherein the light source comprises a first and a second incandescent light;
wherein the first wavelength of light is generated by the first incandescent light coupled to a first optical filter which filters the first light projection produced by the first incandescent light to produce the first wavelength of light; and
wherein the second wavelength of light is generated by the second incandescent light coupled to a second optical filter which filters the second light projection produced by the second incandescent light to produce the second wavelength of light.

40. The method of claim 32 wherein the first and second light projections pass through the tissue of the subject and the light detector is positioned so that the first and second light projections transmitted by the light source are transmitted in a straight line from the light source through the tissue of the subject to the light detector.

41. The method of claim 32 wherein the first and second light projections are reflected by the tissue of the subject and are received by the light detector after being reflected by the tissue of the subject.

42. The method of claim 32 wherein the first and second light projections are received by collection optics which direct the first and second light projections after they has passed through tissue of a subject and deliver it to the light detector, the collection optics selected from the group consisting of lenses or fiber optic cables.

43. A method of determining a tissue hydration value of a subject comprising:
providing a light source which provides a light projection having an intensity toward a tissue of the subject;
providing diffraction optics which diffract the light projection produced by the light source after it has passed through, or reflected off, the tissue of the subject, the diffracted light being directed to a light detector, the diffraction optics selected from the group consisting of prisms, slits, and diffraction gratings;
the light detector detecting at least two intensities of the light projection after the light projection has passed through the diffraction optics; and
the light detector transmitting the at least two intensities of the light projection to a fluid calculation system comprising a microprocessor that is programmed with a tissue hydration model, wherein the tissue hydration model comprises a look-up table including a calibration data set correlating a set of R values to a set of tissue hydrations values;
the microprocessor determining at least two amounts of light transmission (T₁ and T₂) through, or off of, the tissue of the subject based on the at least two intensities from the light detector;
the microprocessor determining an R value where R=T₁/T₂; and
comparing the R value to the tissue hydration model to determine the tissue hydration value.
